(19) United States
(12) Patent Application Publication
(10) Pub. No.: US 2024/0047039 A1
(43) Pub. Date: Feb. 8, 2024

(54) SYSTEM AND METHOD FOR CREATING A CUSTOMIZED DIET

(71) Applicant: Taehoon YOON, Seoul (KR)

(72) Inventor: Taehoon YOON, Seoul (KR)

(21) Appl. No.: 18/206,229

(22) Filed: Jun. 6, 2023

(30) Foreign Application Priority Data
Aug. 2, 2022 (KR) 10-2022-0095832

(51) Int. Cl. G16H 20/60; G09B 19/00; G16H 40/67

(52) U.S. Cl. CPC G16H 20/60; G09B 19/0092; G16H 40/67; A61B 5/4806

(57) ABSTRACT

A system for creating a customized diet menu includes (a) a user terminal obtaining user data, and (b) a server including data collection module, an analysis module, a diet creation module, and a feedback module, wherein the data collection module that collects the user data, food nutritional information, and user input data, wherein the analysis module analyzes user's physical condition changes depending on meal taken by a user and generates user's physical condition change information , wherein the diet creation module creates (i) a basic menu using the food nutritional information and the user input data, (ii) a first user-specific menu using the user data, and a second user-specific menu using the user's physical condition change information , wherein the feedback module feedbacks the user's physical condition change information from the analysis module to the diet creation module.

### CROSS-REFERENCE TO RELATED APPLICATION

This application claims the benefit of priority to Korean Patent Application No. 10-2022-0095832 filed on Aug. 2, 2022, which is herein incorporated by reference in its entirety.

### TECHNICAL FIELDS

The present disclosure relates to a system and method for creating a customized diet menu or meal. More specifically, the present invention relates to a system and method for creating and a customized diet menu or meal by monitoring dietary habits and body weight of a user and then preparing and providing a user-specific menu or meal to a user.

“Diet” is an English word that means a meal plan designed for a specific purpose.

Diet is a steady interest of many people, and as more people have gained weight during the recent pandemic era, interest in diet is growing.

Above all, the food delivery industry is growing, and instant food and high-calorie food are more easily accessible at home. So there is a great demand for diet management that can improve health and help weight loss.

Diet and weight loss through diet management are generally recognized as very difficult because people continue to eat the foods they normally eat even during diet, and because it is difficult for people to obtain information on healthy foods and diet information suitable for their changing body condition.

(Patent Document 0001) 1. Korean Patent Registration No. 10-2371787 (2022.03.03)

(Patent Document 0002) 2. Korean Patent Registration No. 10-2130772 (2020.06.30)

### Problems to be Solved

A system and method for creating a customized diet according to an embodiment generates a customized diet by monitoring each user's dietary habits and body weight and prepares and provides a meal based on the created diet to the user.

In an embodiment, to track user's weight change before and after eating, the system collects (i) the user data and (ii) delivery status. The delivery status tracks whether a meal is successfully delivered to a user. The system analyzes correlation between the user data and a meal which the user takes, and then creates a user specific menu suitable for user's weight and user's physical condition. The user data includes (i) weight before and after eating, (ii) weight before and after sleep, (iii) sleep pattern including sleep hours, sleep time, and sleep state, and (iv) a physical condition of a user.

In an embodiment, (i) user data and (ii) user-specific diet (i.e., meal plan) or user-specific menu, which is created using the user data, are stored and accumulated in the system so that an automatic meal order can be performed. The automatic meal order is specifically designed in consideration of the user's weight change.

In addition, in an embodiment, a correlation between diet (i.e., a meal plan), sleep, and weight of a given user is identified based on a difference between weight of a given day's morning and weight of the previous night to create a customized diet (i.e., a meal plan) in consideration of the weight, hours of sleep, and physical condition of the given user. In addition, changes in user's physical condition after food intake are monitored to update a user-specific diet or a user-specific menu.

### SUMMARY OF INVENTION

In an embodiment, a system for creating a customized diet menu includes a user terminal and a server. The user terminal obtains user data. The user data includes (i) weight before and after eating, (ii) weight before and after sleep, (iii) sleep pattern including sleep hours, sleep time, and sleep state and (iv) a physical condition of a user. The server includes: (a) a data collection module; (b) an analysis module; (c) a diet creation module; and (d) a feedback module.

The data collection module collects the user data, food nutritional information, and user input data. The food nutritional information includes (i) basic nutrients and calories required according to gender, weight, age, and physical specificity, and (ii) nutrients and calories per serving contained in a given food. The user input data includes (i) user's food preference, and (ii) user's diet goal.

The analysis module receives the user data, the food nutritional information, and the user input data from the data collection module; analyzes correlation between (i) the user's weight, (ii) food type and amount, and (iii) user's weight change; selects food type and amount suitable to control user's weight; analyzes user's physical condition change upon intake of food by a user; and generates user's physical condition change information. The user's physical condition change information includes (i) changes in weight before and after eating, (ii) changes in weight before and after sleep, and (iii) changes in sleep pattern including sleep hours, sleep time, and sleep state.

The analysis module (i) defines a difference between a previous night's weight and a previous morning's weight as a day difference value, (ii) defines a difference between the previous night's weight and today morning's weight as a sleep effect value, and (iii) defines a difference between the today morning's weight and the previous morning's weight as a weight change value. The analysis module (i) determines that weight is gained when the day difference value is greater than the sleep effect value, (ii) determines that there is no change in weight when the day difference value and the sleep effect value are the same as each other, and (iii) determines that weight is lost when the day difference value is less than the sleep effect value.

The diet creation module creates a basic menu, a first user-specific menu, and a second user-specific menu. The diet creation module creates the basic menu using (i) the food nutritional information, (ii) the user input data, or (iii) both each of which is received from the data collection module. The diet creation module creates the first user-specific menu by changing the basic menu using the user data received from the data collection module. The diet creation module creates the second user-specific menu by changing the first user-specific menu by using the user's physical condition change information received from the analysis module.

The feedback module (i) tracks the user's physical condition change upon intake of food by the user and (ii) feedbacks the user's physical condition change information to the analysis module.

The server further includes a delivery module. Upon request for the first or the second user specific menu, the delivery module arranges delivery of the first or the second user specific menu to the user.

The data collection module (i) collects the user data from multiple users, (ii) classifies the user data by gender, age, and weight, and (iii) accumulates the user data using the user's physical condition change information.

In an embodiment, the server further comprises a delivery route creation module. The delivery route creation module creates an optimal delivery route according to (i) a delivery destination and (ii) a desired delivery time.

In an embodiment, a method for creating a customized diet menu includes the following (a)-(f) steps. In step (a), the method collects user data, food nutritional information, and user input data. The user data includes (i) weight before and after eating, (ii) weight before and after sleep, (iii) a sleep pattern including sleep hours, sleep time, sleep state, and (iv) a physical condition of a user. The food nutritional information includes (i) basic nutrients and calories required according to gender, weight, age, and physical specificity, and (ii) nutrients and calories per serving contained in a given food. The user input data includes (i) user's food preference, and (ii) user's diet goal.

In step (b), the method receives the user data, the food nutritional information, and the user input data from the data collection module; analyzes correlation between (i) the user's weight, (ii) food type and amount, and (iii) user's weight change; selecting food type and amount suitable to control user's weight; and analyzes user's physical condition change depending on food taken by the user and generates user's physical condition change information.

The user's physical condition change information includes (i) changes in weight before and after eating, (ii) changes in weight before and after sleep, and (iii) changes in sleep pattern including sleep hours, sleep time, and sleep state.

The step of analyzing user's physical condition changes includes: (i) defining a difference between a previous night's weight and a previous morning's weight as a day difference value, (ii) defining a difference between the previous night's weight and today morning's weight as a sleep effect value, (iii) defining a difference between the today morning's weight and the previous morning's weight as a weight change value, (iv) determining that weight is gained when the day difference value is greater than the sleep effect value, (v) determining that there is no change in weight when the day difference value and the sleep effect value are the same as each other, and (vi) determines that weight is lost when the day difference value is less than the sleep effect value.

In step (c), the method creates a basic menu, a first user-specific menu, and a second user-specific menu. The basic menu is created using (i) the food nutritional information, (ii) the user input data, or (iii) both. The first user-specific menu is created by modifying the basic menu using the user data. The second user-specific menu is created by modifying the first user-specific menu by using the user's physical condition change information.

In step (d), the method provides feedback the user's physical condition change information to create the second user-specific menu.

In step (e), upon request for the first or the second user specific menu, the method arranges delivery of the first or the second user specific menu to the user; and

In step (f), the method collects the user data from multiple users; classifies the user data by gender, age, and weight; and accumulates the user data using the user's physical condition change information.

### Advantages of the Invention

The system and the method according to the present invention can greatly improve a user's diet effect and health in a short period of time by monitoring user data and creating and providing a customized diet or a user-specific menu based on the collected user data. The user data includes (i) weight before and after eating, (ii) weight before and after sleep, (iii) sleep pattern including sleep hours, sleep time, and sleep state, and (iv) physical condition of a user.

In addition, the system and method according to the present invention can maintain the dieting effect by (i) monitoring user's physical condition change information after food intake and (ii) updating the customized diet or the user-specific menu based on the user's physical condition change information.

The advantages of the present invention are not limited to the above-mentioned effects and should be understood to include any effects obtainable from the present invention.

### BRIEF DESCRIPTION OF DRAWINGS

FIG. 1 shows the configuration of a system for creating a customized diet (also referred to as a user-specific menu) according to an embodiment.

FIG. 2 shows a data processing configuration of a server (200) according to an embodiment.

FIG. 3 shows a data processing process for providing a customized diet according to an embodiment.

FIG. 4 through FIG. 6 show interfaces for inputting user data according to an embodiment.

FIG. 7 shows interfaces for providing user's physical condition change information according to an embodiment.

FIG. 8 through FIG. 10 show guide interfaces of a system providing a customized diet or a user-specific menu according to an embodiment.

### DETAILED DESCRIPTION

Advantages and features of the present invention, and methods for achieving them, will become clear with reference to the embodiments described below in detail in conjunction with the accompanying drawings. However, the scope of the present invention is not limited to the embodiments disclosed below. Instead, the present invention may be implemented in various different forms. The present embodiments are provided merely to disclose the present invention as complete as possible and help a person having an ordinary skill in the art understand this invention. It should be noted that the scope of the present invention should be determined by the claims. Like reference numerals designate like elements throughout the specification.

In describing the embodiments of the present invention, description on a known function or configuration or structure may be omitted if such description is found unnecessary or such description may hinder concise explanation of the present invention.

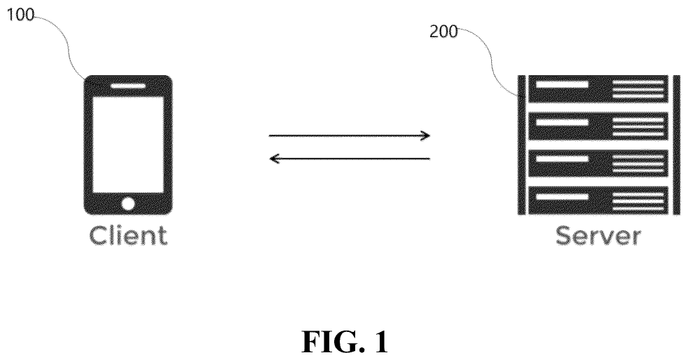

FIG. 1 shows the configuration of a system for creating a customized diet or a user-specific menu according to an embodiment.

Referring to FIG. 1, a system for creating a customized diet according to an embodiment may include a user terminal (100) and a server (200). The user terminal (100) acquires user data and transmits the obtained user data to the server (200). The user data includes (i) weight before and after eating, (ii) weight before and after sleep, (iii) sleep pattern including sleep hours, sleep time, and sleep state, and (iv) physical condition of a user. The server (200) collects food nutritional information. The food nutritional information includes (i) basic nutrients and calories required according to gender, weight, age, and physical specificity such as diathesis, and (ii) nutrients and calories per serving contained in a given food.

The server (200) then creates a basic diet (i.e., meal plan) or a basic menu based on the collected data.

The server (200) analyzes the user data and changes the basic diet or a basic menu to a user-specific diet using the user data analysis result.

In an embodiment, upon receiving a request for ordering the user-specific diet, a meal or a menu prepared according to the user-specific diet (i.e., meal plan) is delivered to the user.

In an embodiment, the server (200) classifies the collected user data by gender, age, and weight.

The server (200) collects and accumulates user's physical condition change information after intake of the meal or the menu and updates the classified user data.

A system and method according to an embodiment creates a customized diet (i.e., meal plan) by monitoring user's eating habits and weight, and provides meals prepared according to the customized meal plan to the user.

In an embodiment, to track user's weight change before and after eating, the system collects (i) the user data and (ii) delivery status. The delivery status tracks whether a meal is successfully delivered to a user. The system analyzes correlation between the user data and a meal which the user takes, and then creates a user specific menu suitable for user's weight and user's physical condition.

The user data includes (i) weight before and after eating, (ii) weight before and after sleep, (iii) sleep pattern including sleep hours, sleep time, and sleep state, and (iv) a physical condition of a user.

In an embodiment, (i) user data and (ii) user-specific diet (i.e., meal plan) or user-specific menu, which is created using the user data, are stored and accumulated in the system so that an automatic meal order can be performed. The automatic meal order is specifically designed in consideration of the user's weight change.

In addition, in an embodiment, a correlation between diet (i.e., a meal plan), sleep, and weight of a given user is identified based on a difference between weight of a given day's morning and weight of the previous night to create a customized diet (i.e., a meal plan) in consideration of the weight, hours of sleep, and physical condition of the given user.

In addition, changes in user's physical condition after food intake are monitored to update a user-specific diet or a user-specific menu.

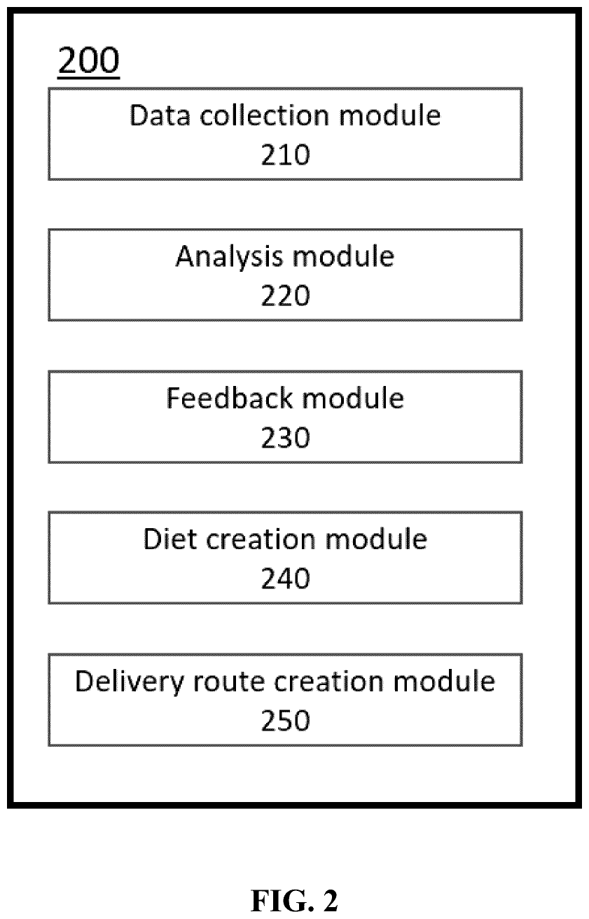

FIG. 2 shows a data processing configuration of a server (200) according to an embodiment.

Referring to FIG. 2, the server (200) according to the embodiment includes a data collection module (210), an analysis module (220), a feedback module (230), a diet creation module (240), and a delivery route creation module (250).

The term ‘module’ used in this specification should be interpreted as being able to include software, hardware, or a combination thereof, depending on the context in which the term is used.

For example, the software may be machine language, firmware, embedded code, and application software.

The hardware may be, for example, a circuit, processor, computer, integrated circuit, integrated circuit core, sensor, micro-electro-mechanical system (MEMS), passive device, or combination thereof.

The data collection module (210) collects user data.

The user data includes (i) weight before and after eating, (ii) weight before and after sleep, (iii) sleep pattern including sleep hours, sleep time, and sleep state, and (iv) a physical condition of a user.

User data is used for monitoring changes in the user's physical condition according to the food ingested.

The data collection module (210) further collects food nutritional information. The food nutritional information includes (i) basic nutrients and calories required according to gender, weight, age, and physical specificity, and (ii) nutrients and calories per serving contained in a given food.

The analysis module (220) analyzes user's physical condition changes depending on meal taken by the user to generate user's physical condition change information.

The user's physical condition change information includes changes (i) in weight before and after eating, (ii) in weight before and after sleep, (iii) in sleep pattern including sleep hours, sleep time, and sleep state, and (iv) in a physical condition of the user.

In an embodiment, the analysis module (220) analyzes the correlation between (i) the user's weight, (ii) food type and amount, and (iii) weight change. The analysis module (220) selects food type and amount suitable to maintain or control user's weight.

In an embodiment, the analysis module (220) (i) defines a difference between a previous night's weight and a previous morning's weight as a day difference value, (ii) defines a difference between the previous night's weight and today morning's weight as a sleep effect value, and (iii) defines a difference between the today morning's weight and the previous morning's weight as a weight change value. See the following Equations 1-3.

The analysis module (220) (i) determines that weight is gained when the day difference value is greater than the sleep effect value, (ii) determines that there is no change in weight when the day difference value and the sleep effect value are the same as each other, and (iii) determines that weight is lost when the day difference value is less than the sleep effect value.

day difference value=user's weight of the previous night−user's weight of the previous morning Equation 1

Sleep effect value=user's weight of the previous night−user's weight of today's morning Equation 2

Weight change value=user's weight of today's morning−user's weight of the previous morning Equation 3

In an embodiment, let's say the weight of the previous night is 71.0 Kg. and the weight of today morning is 70.4 Kg. Since the weight of the previous night is greater than the weight of today's morning, the sleep effect value is calculated by subtracting the weight of today's morning from the weight of the previous night. Thus, the sleep effect value is calculated to be 71.0−70.4=0.6.

In an embodiment, the day difference value and the sleep effect value are compared in a state of positive (+) values.

In an embodiment, the analysis module (220) (i) collects user activity amount information including the number of walking steps for a certain period of time and (ii) calculates the user's activity amount for a given period, so that the activity amount information can be considered to create a user-specific diet or menu.

For example, the analysis module (220) may determine a certain percentage of an average number of the user's walking steps for a week as the user's activity amount, and consider the user's activity amount information to calculate the total amount of calories contained in a meal provided to the user.

In addition, when the day difference value is greater than a first given ratio (e.g. 1.5%) of the morning weight value, the analysis module (220) determines that it is binge eating and weight gain. When the day difference value is more than a second given ratio (e.g. 1.0%) of the weight in the morning and is less than the first given ratio (e.g. 1.5%) of the morning weight , the analysis module (220) determines that it is overeating and weight gain; When the day difference value is more than a third given ratio (e.g. 0.5%) of the weight in the morning and is less than the second given ratio (e.g. 1.0%) of the morning weight, the analysis module (220) determines that it is normal or maintaining weight. When the day difference value is less than the third given ratio (e.g. 0.5%) of the morning weight, the analysis module (220) determines that there is weight loss. See the Equations 4 to 7, shown below.

In the case of morning weight*1.5%≤day difference value, binge eating or weight gain Equation 4

Overeating or weight gain in case of morning weight*1.0%≤day difference value<morning weight*1.5% Equation 5

In the case of morning weight*0.5%≤day difference value<morning weight*1.0%, normal weight Equation 6

Eating less or Weight loss in case of morning weight*0.5%>day difference value Equation 7

In the embodiment, the analysis module (220) can determine how many times of a weight loss event should be repeated to reduce user's weight to a given weight loss target. For example, the weight loss event can be set to keep the daily value to be 0.5% or less of the user's current weight. The weight loss event can be adjusted depending on the weight target.

In the embodiment, the system notifies the user of the number of weight loss events necessary to achieve the weight loss target and counts the number of achievement event whenever the user successfully performs the weight loss event.

In another example, the weight loss event can be set to (i) eat lightly, (ii) weight loss, or (iii) keeping weight as is. The analysis module (220) may (i) count how many number of the weight loss events is successfully performed during a preset time period, and (ii) set a given number of the weight loss event successfully performed to the weight loss target.

In an embodiment, the weight loss target may be set to count, during seven (7) days, (i) a positive event 4 times or more and (ii) a negative event 3 times or less. The positive event may include eating lightly, happening of weight loss, or keeping weight as is, etc. The negative event may include happening of weight gain.

In an embodiment, the weight target or a diet goal may be set and adjusted by a user.

The analysis module (220) may calculate meal calories and types for a user and adjust the meal calories and types depending on user's weight change and user's weight target.

The feedback module (230) tracks how user's physical condition, such as user's weight, changes upon intake of the meal, and feedbacks user's physical condition change information from the analysis module to the diet creation module.

The user's physical condition change information includes (i) changes in weight before and after eating, (ii) changes in weight before and after sleep, and (iii) changes in sleep pattern including sleep hours, sleep time, and sleep state.

Upon receiving the user's physical condition change information from the feedback module (230), the analysis module (220) adjusts the meal calories, types, amounts, etc. that is provided to the user in consideration of the user's physical condition change information, such as changes in user's weight.

The diet creation module (240) may update or adjust user-specific menu by using the user's physical condition change information.

The analysis module (220) receives the user data, the food nutritional information, and the user input data from the data collection module; analyzes correlation between (i) the user's weight, (ii) food type and amount, and (iii) user's weight change; selects food type and amount suitable to control user's weight; analyzes user's physical condition change upon intake of food by a user; and generates user's physical condition change information. The analysis module (220) may employ artificial intelligence technology or machine learning technology to calculate the meal calories, types, amounts, etc. necessary for a user to achieve the diet goal.

In an embodiment, the analysis module (220) is configured to handle noises. In addition, the analysis module (220) may process unlearned patterns through out-of-distribution data detection.

The out-of-distribution data detection determines whether an image which is input to artificial intelligence is learned probability distribution data or not.

In an embodiment, the out-of-distribution data detection can improve stability and reliability of the system by (i) filtering out images that are difficult for an artificial neural network to process or (ii) classifying the images as exceptions.

In an embodiment, the out-of-distribution data detection can be performed by calibrating a probability value which indicates how much reliable a deep learning decision is. Credibility of the out-of-distribution data detection can be improved by generating the out-of-distribution data using a generative adversarial network (GAN) and by making the system learn the out-of-distribution data.

In an embodiment, in order to reduce the size of the model while maintaining credibility of user data analysis, the user-specific menu can be generated using lightweight deep learning technology. The lightweight deep learning technology is advantageous in simplifying calculation.

In an embodiment, data light-weighting is preferred for an effective user data analysis. Data light-weighting can be performed by reducing an operation dimension, by pruning, or by quantization. The reduction of the operation dimension can be performed by modifying a convolution filter in a Convolution Neural Network (CNN).

The pruning can be performed by deleting a weight value of a less significant neural network. The quantization can be performed by reducing the floating point of a weight value to simplify calculation.

In an embodiment, simplified operation and improved credibility can be obtained (i) by making a large neural network learned in advance and (ii) then by training a small neural network to imitate the output of the large neural network.

The menu generation module (240) creates a basic menu, a first user-specific menu, and a second user-specific menu. The first and the second user-specific menus are collectively referred to as a “user-specific menu.”

The basic menu is created using the user input data. The user input data includes user's food preference and a user's diet goal.

The user-specific menu is created using the user's physical condition change information.

The delivery route creation module (250) creates an optimal delivery route according to (i) a delivery destination and (ii) a desired delivery time.

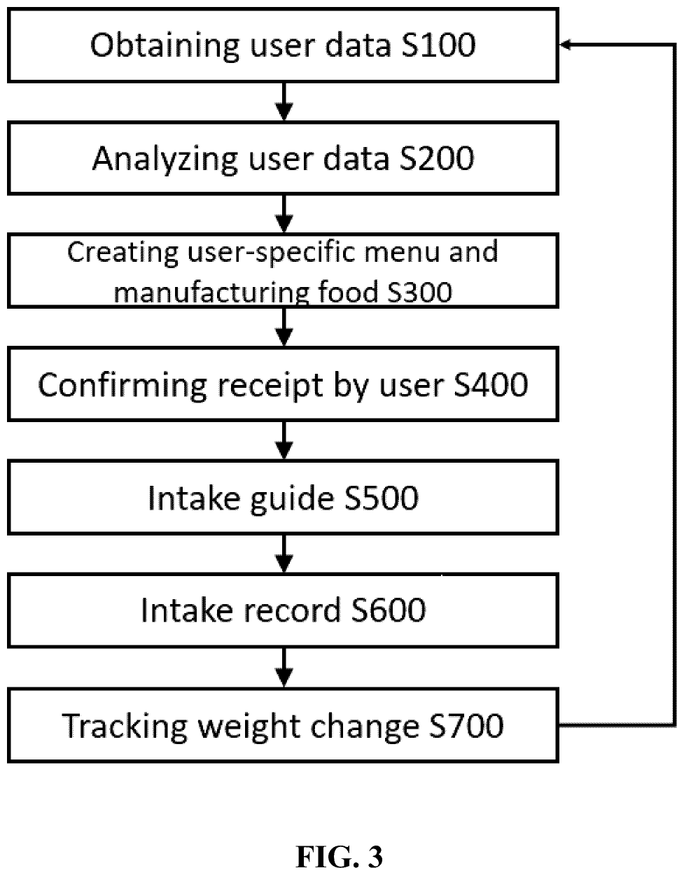

FIG. 3 is a diagram showing how to process data for providing a customized diet according to an embodiment.

Referring to FIG. 3, in step S100, user data is collected.

The user data includes (i) weight before and after eating, (ii) weight before and after sleep, (iii) a sleep pattern, (iv) a physical condition of a user, and (v) amount of activity of the user. The sleep pattern includes sleep hours, sleep time, sleep state.

In step S200, user data is analyzed.

In step S300, a user-specific menu is created using an analysis result obtained in step S200.

In step S400, it is confirmed whether the user receives the user-specific menu. In step S500, when it is confirmed that the user receives the user-specific menu, information or a guideline on the user-specific menu delivered is provided.

In step S600, user's intake of the user-specific menu delivered is recorded. In step S700 user's weight change is tracked.

After Step 700, the process returns to step S100 to feedback the user's weight change into the system to modify the user-specific menu.

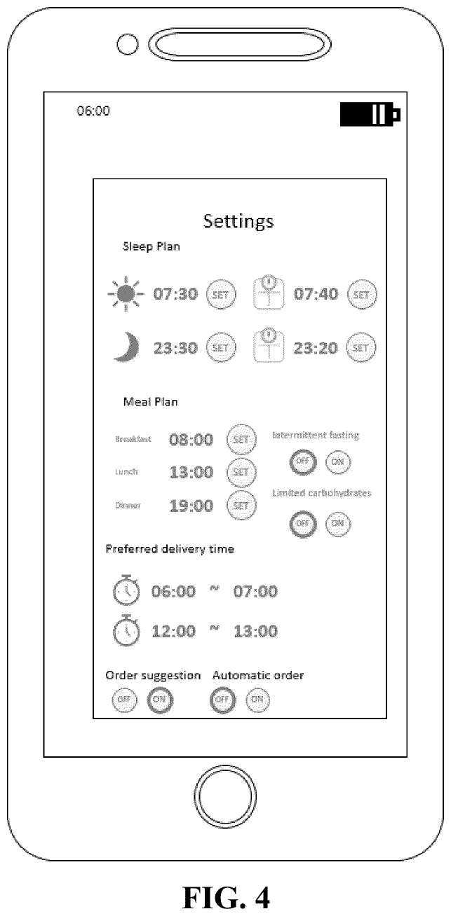

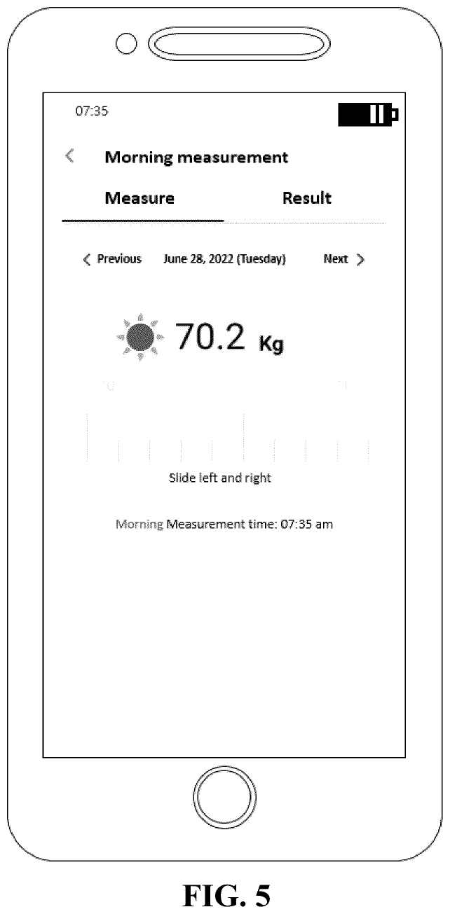

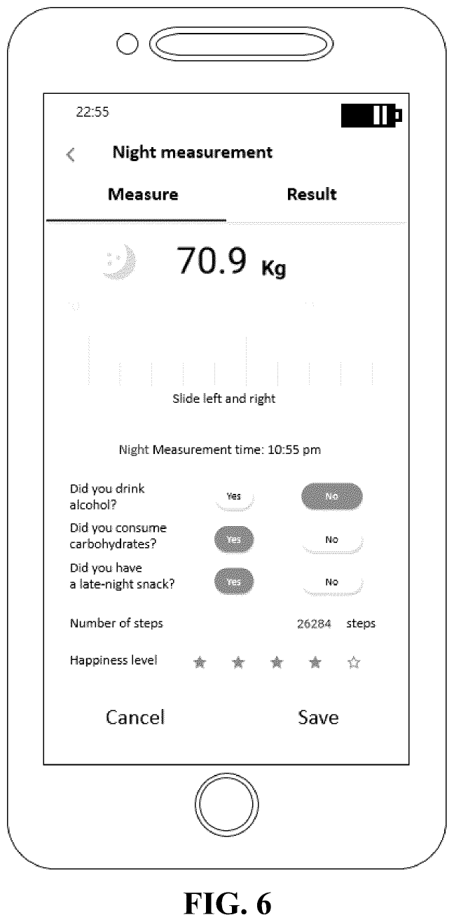

FIGS. 4 to 6 are diagrams illustrating an interface for receiving user input data. FIGS. 4-6 are diagrams illustrating an interface for receiving user input data.

Referring to FIG. 4, in an embodiment, user input data is obtained through a user terminal. The user input data may include sleep plan, meal plan, preferred delivery time, morning weight, night weight, meal habit information, and activity information of the user.

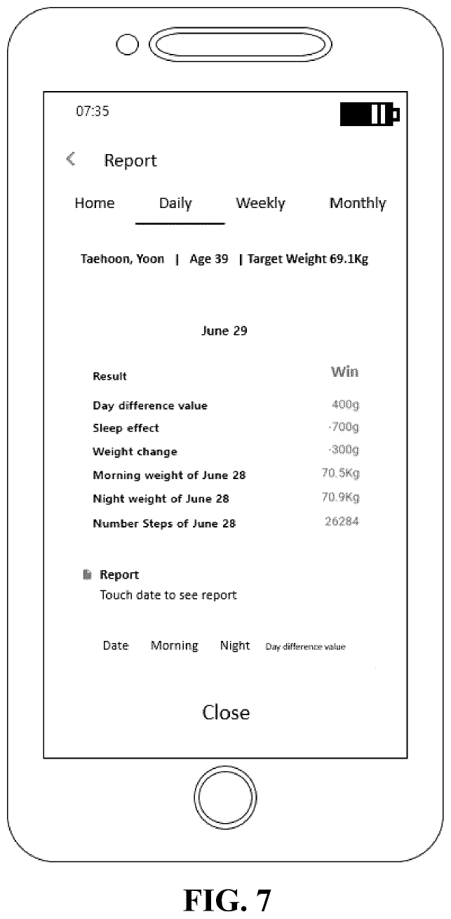

FIG. 7 is a diagram illustrating interfaces for analysis results which are obtained by analyzing user data.

The user data may include (i) weight before and after eating, (ii) weight before and after sleep, (iii) a sleep pattern including sleep hours, sleep time, sleep state, and (iv) a physical condition of a user. The food nutritional information includes (i) basic nutrients and calories required according to gender, weight, age, and physical specificity, and (ii) nutrients and calories per serving contained in given food.

Referring to FIG. 7, (i) weight change information, (ii) day difference value, and/or (iii) sleep effect, etc. can be displayed on the user terminal. The weight change information may be indicated as weight gain, no change in weight, or weight loss.

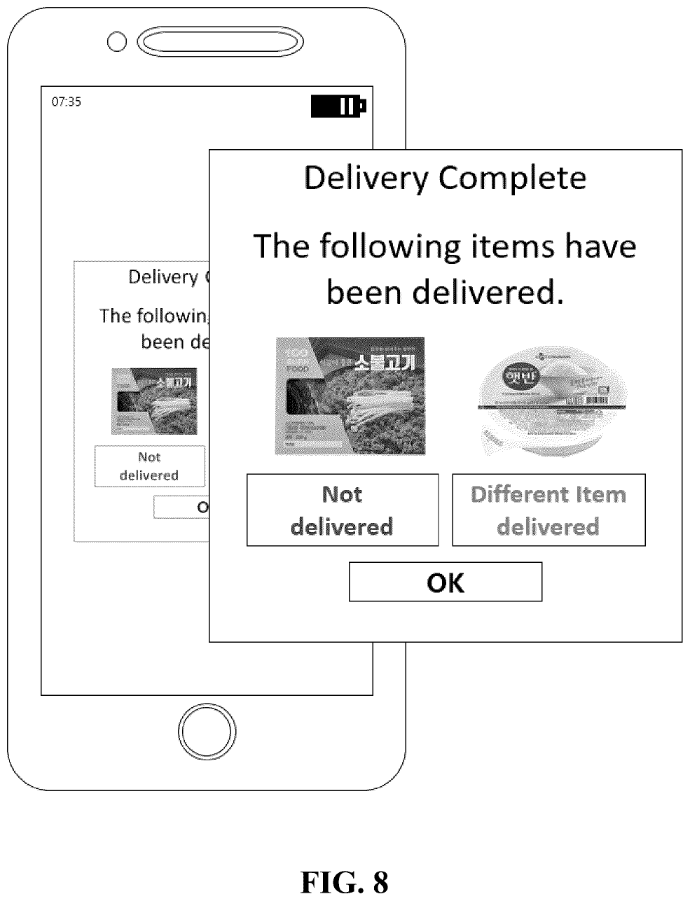

FIGS. 8 through 10 are guide interfaces of a system providing a customized diet according to an embodiment.

Referring to FIG. 8, the system provides a notice when food or meal is delivered. The food or meal delivered is prepared according to a user-specific diet.

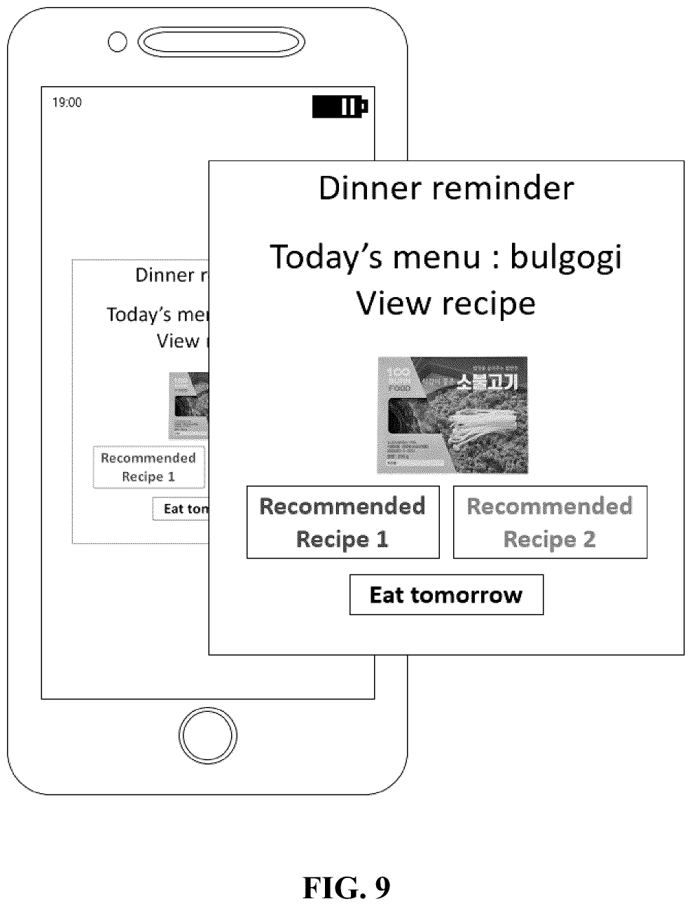

Referring to FIG. 9, recipe information of food or meal delivered may be provided.

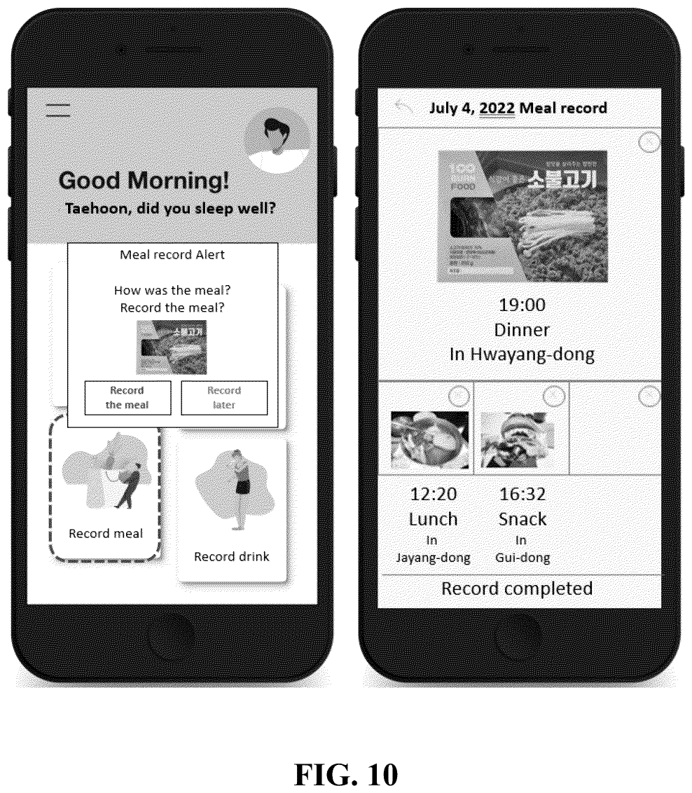

Referring to FIG. 10, a meal record interface is provided to collect user's intake information. For example, after completion of a meal, the user can enter what she/he took through the meal record interface. The user's intake information can be provided in a timeline format.

The customized diet generation system and method as described above (i) monitors user data such as weight, physical condition, and sleep patterns, and (ii) provides customized diet information based on the collected user data, thereby improving the user's diet effect and health in a short period of time.

In addition, by monitoring a user's physical condition change after food intake and generating a user-specific diet based on feedback of the user's physical condition change, the system and method may help the user to maintain a diet effect.

Since the disclosed content is only an example and can be variously modified and implemented by a person having an ordinary skill in the art without departing from the gist and the scope of the patent claims, the protection scope of the disclosed invention is determined by the claims and is not limited to one particular embodiment.

What is claimed is:

1. A system for creating a customized diet, comprising:
a user terminal and a server,
wherein the user terminal obtains user data,
wherein the user data includes (i) weight before and after eating, (ii) weight before and after sleep, (iii) sleep pattern including sleep hours, sleep time, and sleep state and (iv) a physical condition of a user,
wherein the server includes: (a) a data collection module; (b) an analysis module; (c) a diet creation module; and (d) a feedback module,
wherein (a) the data collection module collects the user data, food nutritional information, and user input data,
wherein the food nutritional information includes (i) basic nutrients and calories required according to gender, weight, age, and physical specificity, and (ii) nutrients and calories per serving contained in a given food,
wherein the user input data includes (i) user's food preference, and (ii) user's diet goal,
wherein (b) the analysis module receives the user data, the food nutritional information, and the user input data from the data collection module; analyzes correlation between (i) the user's weight, (ii) food type and amount, and (iii) user's weight change; selects food type and amount suitable to control user's weight; analyzes user's physical condition change upon intake of food by a user; and generates user's physical condition change information,
wherein the user's physical condition change information includes (i) changes in weight before and after eating, (ii) changes in weight before and after sleep, and (iii) changes in sleep pattern including sleep hours, sleep time, and sleep state,
wherein the analysis module (i) defines a difference between a previous night's weight and a previous morning's weight as a day difference value, (ii) defines a difference between the previous night's weight and today morning's weight as a sleep effect value, and (iii) defines a difference between the today morning's weight and the previous morning's weight as a weight change value,
wherein the analysis module (i) determines that weight is gained when the day difference value is greater than the sleep effect value, (ii) determines that there is no change in weight when the day difference value and the sleep effect value are the same as each other, and (iii) determines that weight is lost when the day difference value is less than the sleep effect value,
wherein (c) the diet creation module creates a basic menu, a first user-specific menu, and a second user-specific menu,
wherein the diet creation module creates the basic menu using (i) the food nutritional information, (ii) the user input data, or (iii) both each of which is received from the data collection module,
wherein the diet creation module creates the first user-specific menu by changing the basic menu using the user data received from the data collection module,
wherein the diet creation module creates the second user-specific menu by changing the first user-specific menu by using the user's physical condition change information received from the analysis module,
wherein (d) the feedback module (i) tracks the user's physical condition change upon intake of food by the user and (ii) feedbacks the user's physical condition change information to the analysis module,
wherein, the server further includes a delivery module,
wherein, upon request for the first or the second user specific menu, the delivery module arranges delivery of the first or the second user specific menu to the user,
wherein (a) the data collection module (i) collects the user data from multiple users, (ii) classifies the user data by gender, age, and weight, and (iii) accumulates the user data using the user's physical condition change information.

2. The system according to claim 1,
wherein the server further comprises a delivery route creation module,
wherein the delivery route creation module creates an optimal delivery route according to (i) a delivery destination and (ii) a desired delivery time.

3. A method for creating a customized diet menu, comprising:
(a) collecting user data, food nutritional information, and user input data,
wherein the user data includes (i) weight before and after eating, (ii) weight before and after sleep, (iii) a sleep pattern including sleep hours, sleep time, sleep state, and (iv) a physical condition of a user,
wherein the food nutritional information includes (i) basic nutrients and calories required according to gender, weight, age, and physical specificity, and (ii) nutrients and calories per serving contained in a given food, wherein the user input data includes (i) user's food preference, and (ii) user's diet goal;
(b) receiving the user data, the food nutritional information, and the user input data from the data collection module; analyzing correlation between (i) the user's weight, (ii) food type and amount, and (iii) user's weight change; selecting food type and amount suitable to control user's weight; and analyzing user's physical condition change depending on food taken by the user and generates user's physical condition change information,
wherein the user's physical condition change information includes (i) changes in weight before and after eating, (ii) changes in weight before and after sleep, and (iii) changes in sleep pattern including sleep hours, sleep time, and sleep state,
wherein the step of analyzing user's physical condition changes includes:
(i) defining a difference between a previous night's weight and a previous morning's weight as a day difference value,
(ii) defining a difference between the previous night's weight and today morning's weight as a sleep effect value,
(iii) defining a difference between the today morning's weight and the previous morning's weight as a weight change value,
(iv) determining that weight is gained when the day difference value is greater than the sleep effect value,
(v) determining that there is no change in weight when the day difference value and the sleep effect value are the same as each other, and
(vi) determines that weight is lost when the day difference value is less than the sleep effect value;
(c) creating a basic menu, a first user-specific menu, and a second user-specific menu,
wherein the basic menu is created using (i) the food nutritional information, (ii) the user input data, or (iii) both,
wherein the first user-specific menu is created by modifying the basic menu using the user data,
wherein the second user-specific menu is created by modifying the first user-specific menu by using the user's physical condition change information;
(d) providing feedback the user's physical condition change information to create the second user-specific menu;
(e) upon request for the first or the second user specific menu, arranging delivery of the first or the second user specific menu to the user; and
(f) collecting the user data from multiple users; classifying the user data by gender, age, and weight; and accumulating the user data using the user's physical condition change information.
